(19) United States
(12) Patent Application Publication
(10) Pub. No.: US 2020/0143922 A1
(43) Pub. Date: May 7, 2020

(54) METHODS AND APPARATUS FOR PREDICTING DEPRESSION TREATMENT OUTCOMES

(71) Applicant: Yale University, New Haven, CT (US)

(72) Inventors: Adam Chekroud, New Haven, CT (US); John H. Krystal, Woodbridge, CT (US); Ralitza Gueorguieva, Gainesville, FL (US); Abhishek Chandra, Brooklyn, NY (US)

(73) Assignees: Yale University, New Haven, CT (US); Spring Care, Inc., New York, NY (US)

(21) Appl. No.: 16/305,468

(22) PCT Filed: Jun. 2, 2017
(86) PCT No.: PCT/US17/35584

§ 371 Date: Nov. 29, 2018

Related U.S. Application Data
(60) Provisional application No. 62/345,701, filed on Jun. 3, 2016.

(51) Int. Cl. G16H 20/10; G16H 10/20; G06N 7/00; G06N 20/00; G06K 9/62

(52) U.S. Cl. CPC G16H 20/10; G16H 10/20; G06N 7/005; G06K 9/6262; G06K 9/6218; G06K 9/6269; G06K 9/6256; G06N 20/00

(57) ABSTRACT

Methods and apparatus for providing a treatment recommendation for a patient having a depression disorder. The method comprising receiving patient information including patient responses to each of a plurality of questions provided on a questionnaire, processing, with a trained statistical model, at least some of the received patient information to determine treatment recommendation information for the patient, and transmitting the treatment recommendation information to an electronic device.

### CROSS-REFERENCE TO RELATED APPLICATIONS

This application claims the benefit of U.S. Provisional Patent Application Ser. No. 62/345,701, filed Jun. 3, 2016, entitled “Treatment Selection for Major Depression Disorder,” the entire contents of which is incorporated by reference herein.

### BACKGROUND

As few as 11-30% of patients with depression reach remission with initial treatment, even after 8-12 months. One factor reducing effectiveness of treatment is the inability to personalize pharmacotherapy. Clinicians match patients with specific antidepressants via a prolonged period of trial and error, delaying clinical improvement and increasing risks and costs of treatment. This potentially leaves patients improperly treated, sometimes at risk of suicide, until they find the appropriate treatment serendipitously. The absence of clinical prediction tools in psychiatry starkly contrasts with other areas of medicine, such as oncology, cardiology, and critical care, where algorithmic models often have important roles in medical decision making, and routinely outperform judgment of individual clinicians.

### SUMMARY

Some embodiments are directed to a system for providing a treatment recommendation for a patient having a depression disorder. The system comprises a server computer configured to receive patient information including patient responses to each of a plurality of questions provided on a questionnaire, process, with a trained statistical model, a set of values determined based, at least in part, on patient information including patient responses to each of a plurality of questions provided on a questionnaire, and transmit the treatment recommendation information to an electronic device.

Other embodiments are directed to a system for training a statistical model used to predict a treatment outcome for patients having depression. The system comprises a server computer configured to determine a set of features to include in the statistical model, train the statistical model using a first labeled dataset of values for the determined set of features, wherein the first labeled dataset includes clinical outcomes and patient information for each of a first plurality of patients, and output the trained statistical model.

Other embodiments are directed to a method of providing a treatment recommendation for a depression disorder. The method comprises processing, using a trained statistical model executing on at least one computer processor, a set of values determined based, at least in part, on patient information including patient responses to each of a plurality of questions provided on a questionnaire, determining treatment recommendation information based, at least in part, on output of the trained statistical model, and transmitting the treatment recommendation information to an electronic device.

Other embodiments are directed to a system for classifying, based on their symptoms, patients having a depression disorder into one or more symptom cluster groups. The system comprises a server computer configured to receive patient responses to each of a plurality of questions provided on a questionnaire by a plurality of patients; apply a symptom clustering technique to the received patient responses to determine a plurality of symptom cluster groups; and output a representation of the plurality of symptom cluster groups.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

Various non-limiting embodiments of the technology will be described with reference to the following figures. It should be appreciated that the figures are not necessarily drawn to scale.

FIG. 1 illustrates an architecture of a computer system within which some embodiments may be used;

FIG. 2 illustrates a flowchart of a process for providing a treatment recommendation for a patient with depression in accordance with some embodiments;

FIG. 3 illustrates a flowchart of a process for training a statistical model in accordance with some embodiments;

FIG. 4 illustrates a portion of a user interface for collecting patient information in accordance with some embodiments;

FIG. 5 illustrates a portion of a user interface for collecting patient demographic information in accordance with some embodiments;

FIG. 6 illustrates a portion of a user interface for collecting patient information in accordance with some embodiments;

FIG. 7 illustrates a portion of a user interface for providing patient instructions in accordance with some embodiments;

FIG. 8 illustrates a portion of a user interface for providing a summary report based on patient information in accordance with some embodiments;

FIG. 9 illustrates a portion of a user interface for providing a summary report based on patient information in accordance with some embodiments;

FIG. 10 illustrates a portion of a user interface for providing treatment recommendations in accordance with some embodiments;

FIG. 11 illustrates a portion of a user interface for providing patient response information in accordance with some embodiments;

FIG. 12 illustrates a portion of a user interface for selecting a questionnaire for collecting patient information in accordance with some embodiments;

FIG. 13 illustrates a portion of a user interface in which patient results are presented in a web browser in accordance with some embodiments;

FIG. 14 illustrates a portion of a user interface in which patient results are presented in a web browser in accordance with some embodiments;

FIG. 15 illustrates a flow chart of a process for developing a statistical model for use with some embodiments;

FIG. 16 illustrates a schematic of cross-trial prediction of antidepressant treatment outcomes in accordance with some embodiments;

FIG. 17 illustrates a flow chart of a process for performing a symptom clustering technique in accordance with some embodiments;

FIG. 18 illustrates a flow chart of a process for developing a statistical model in combination with a symptom clustering technique for use with some embodiments;

FIG. 19 illustrates groupings of symptoms based on a hierarchical clustering technique in accordance with some embodiments;

FIG. 20 illustrates plots for model-fitted outcome trajectories for symptom clusters in accordance with some embodiments; and

FIG. 21 illustrates a schematic of results for using a statistical model to predict treatment outcomes specific for a symptom cluster in accordance with some embodiments.

### DETAILED DESCRIPTION

When a patient presents seeking treatment for depression, they are typically prescribed a course of antidepressants. Most patients do not reach remission when they take the first antidepressant. However, there are a large number of antidepressants available for selection, and most patients eventually find a treatment that works for them through trial and error. This period of trial and error is costly both in terms of patient health and also costly in terms of unnecessary treatment expense. Accordingly, it would be desireable to have a tool that recommends a treatment protocol for a patient that is likely to be effective for that patient as soon as possible, to reduce harm and discomfort and provide substantial reductions in cost.

The inventors have recognized and appreciated that conventional trial and error approaches to matching a patient with a specific antidepressant treatment may be improved by using data-driven techniques to provide treatment recommendations for patients with depression. Accordingly, some embodiments are directed to a trained computer-implemented predictive model that takes as input patient characteristics and provides as output personalized treatment recommendations for the patient.

Algorithmic tools that predict treatment outcomes in depression may be useful for a number of parties. Healthcare agencies may seek to use them to determine whether predicted non-response to a medication justifies the allocation of funds to support more expensive psychotherapies. Insurance companies may consider predicted outcomes when deciding whether to cover a certain drug prescription: a 12-week course of the antidepressant Celexa costs around $450-500 and it may be both cheaper and more effective to allocate predicted non-responders to alternative antidepressant treatments. For an individual patient, models designed in accordance with some embodiments reduce time spent taking ineffective treatments, give patients greater insight into their likely illness course, and thus can reduce harm and illness duration overall.

A challenge when developing predictive clinical tools is to establish what information should be used. Genetic and brain imaging measures are possible sources of information, and have generated interest. However, even if effective, the cost and time of collecting and processing data is often not practical. By contrast, behavioral (e.g., patient-reported) data are routinely collected as part of a patient visit. Clinical experience guides what information is used in treatment decisions; however, early-stage clinicians have little experience, and even experienced clinicians might overlook useful information or overweight salient clinical examples. Previous attempts to identify clinical predictors of treatment outcome have generally identified a few predictors based on clinical experience, and have investigated their overall effect in a stepwise manner. Another approach quantified the effect of nine symptom dimensions derived from a factor analysis.

The inventors have recognized and appreciated that examination of all potential predictors simultaneously in an unbiased manner provides an opportunity for discovery of the most predictive feature. Machine learning techniques are especially well suited for this challenge. Rather than separately considering the effect of one variable on an outcome of interest, machine-learning techniques identify patterns of information in data that are useful to predict outcomes at the individual level. Modern machine learning approaches offer benefits over traditional statistical approaches (generalized linear models, and even non-linear regression models [generalized additive models]) due to their ability to detect complex (e.g., non-linear) high-dimensional interactions that might inform predictions.

Some embodiments are directed to a computer-implemented system that employs a machine learning technique to predict treatment outcomes for a patient based, at least in part, on patient information provided as input to a trained model. As discussed in further detail below, some implementations present a questionnaire to a patient on an electronic device, such as a tablet computer or smartphone when they arrive at a healthcare provider's office. At least some the patient information is provided as input to a statistical model trained on labelled data to assess the likelihood that a course of treatment on a particular antidepressant will be effective for the patient.

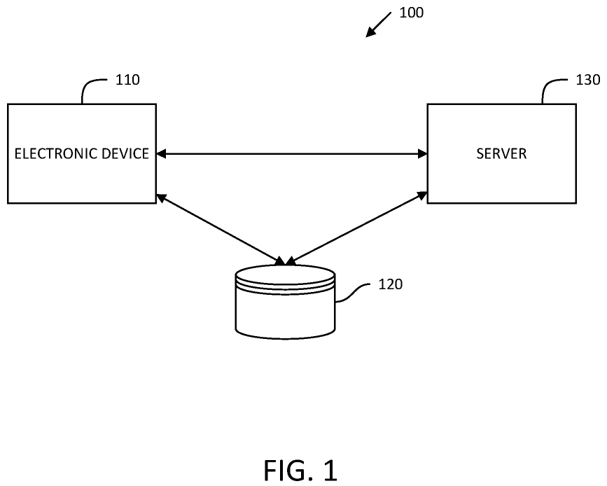

FIG. 1 schematically illustrates an exemplary architecture of a computer system architecture 100 on which some embodiments may be implemented. Computer system 100 includes an electronic device 110 configured to provide an interface that enables a patient to provide information about their medical/psychiatric background and/or current symptom profile. In some embodiments, electronic device 110 is a portable electronic device such as a smartphone, tablet computer, or laptop computer configured to execute an application that provides a user interface displaying a questionnaire with which a patient may interact to provide the patient information. The questionnaire may be provided by a dedicated application executing on electronic device 110 or alternatively the questionnaire may be provided within a more generalized application such as a web browser, and embodiments of the invention are not limited in this respect. Examples of each of these types of implementations is described in more detail below.

Computer system 100 also includes datastore 120 and server 130. Datastore 120 is configured to store data received from electronic device 110, such as patient information provided by a patient via a user interface, and/or data received from server 130, such as information output from one or more predictive models executing on server 130, examples of which are described in more detail below.

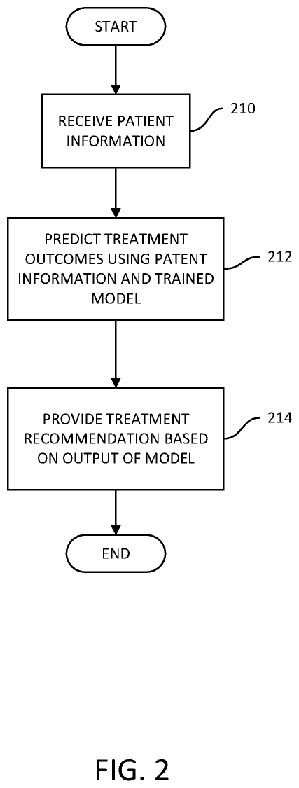

FIG. 2 shows a flowchart of a process for providing a treatment recommendation for a patient with depression in accordance with some embodiments. In act 210, patient information is received via a user interface, examples of which are described in more detail below. In some embodiments, a user interface provided on a portable electronic device such as a smartphone or tablet computer may display a plurality of questions in a questionnaire and a patient may interact with the user interface to provide responses to the plurality of questions. The user interface may also be configured to collect patient demographic information and/or other patient medical history information.

The process then proceeds to act 212, where at least some of the received patient information is used in combination with a trained statistical model to predict treatment outcomes for a patient if treated with a particular antidepressant or combination of antidepressants. In some embodiments, described in more detail below, at least some of the patient information is provided as input to the trained statistical model that provides as output treatment outcome information based on the patient information provided as input. In some embodiments, the patient information comprises a plurality of computer-readable values derived from a patient's responses to a plurality of questions provided on a questionnaire. The plurality of computer-readable values may correspond, for example, to a set of input features for the trained statistical model. In some embodiments, at least some of the received patient information is processed using a symptom clustering technique and the one or more predicted treatment outcomes are based, at least in part, on an output of the symptom clustering process. In some embodiments, the received patient information may be processed to place the patient information into a format acceptable as input to the trained statistical model. For example, the patient information may converted to a plurality of computer-readable values (e.g., binary or other numerical values) corresponding to a set of features associated with the model.

The process then proceeds to act 214, where the one or more predicted treatment outcomes are provided as output of the model. The output of the model may comprise a plurality of values representing a percentage likelihood that a treatment will be effective in treating the patient. For example, in some embodiments the output of the model is a plurality of values, e.g., ranging from −1 to 1, that are interpreted to provide a treatment recommendation for the patient.

In some embodiments, a treatment recommendation may be displayed to a healthcare provider of the patient based on the analysis by the trained statistical model to help the healthcare provider make an informed decision about which antidepressant or combination of antidepressants to prescribe the patient based on the likelihood that such a course of medication would yield favorable results for the patient. For example, the one or more predicted treatment outcomes may include a recommendation to treat the patient with one or more antidepressants including, but not limited to, citalopram, escitalopram, and a combination of escitalopram and bupropion.

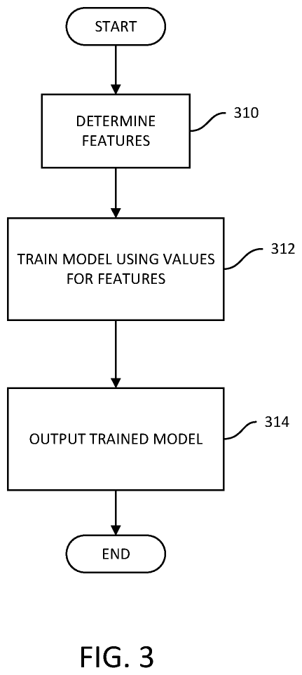

As discussed above, some embodiments are directed to techniques for using a trained statistical model to predict treatment outcomes for a patient with depression. FIG. 3 illustrates a flowchart of a process for training a statistical model in accordance with some embodiments. In act 310, input features for the statistical model may be determined. An example of determining input features for a statistical model is described in more detail below. For example, in some embodiments, the set of features may be determined by applying a penalized logistic regression to a “labelled dataset” that specifies values for each of plurality of features to determine the N-most salient predictors. The process then proceeds to act 312, wherein the statistical model is trained using labelled data that specifies values for each of the input features. Using labeled data enables the model to learn a set of weights between the nodes in the model that result in a particular outcome. As discussed in more detail below, the trained statistical model may optionally be validated using a second dataset that is different from the first dataset to ensure that the model is generalizable beyond just the training data.

The process then proceeds to act 314, where the trained statistical model is output for use in predicting treatment outcomes in accordance with some embodiments. The model may be initially trained based on a large dataset of labelled data and the model may be retrained during use of the model as new data is input and classified by the model to refine the weights in the model.

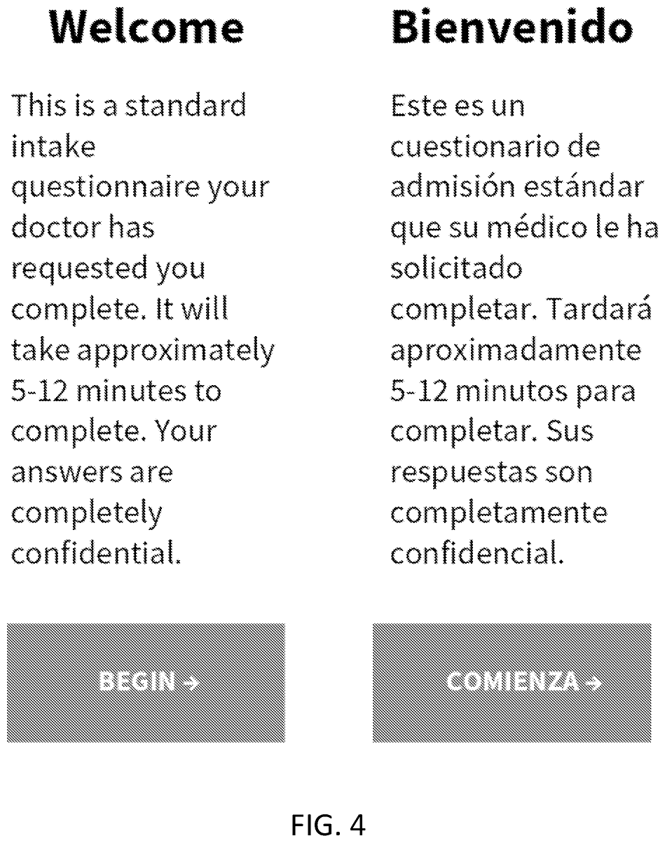

FIGS. 4-14 illustrate portions of user interface that may be used in accordance with some embodiments to collect patient information used in predicting treatment outcomes in patients with depression and/or may be used to present information about the predicted treatment outcomes to a patient and/or a healthcare provider of the patient to facilitate a treatment evaluation process.

FIG. 4 illustrates an introduction screen portion of a user interface that may be presented to a patient on a tablet computer upon arrival to a healthcare provider's office. As shown, the introduction screen provides information and instructions regarding a questionnaire that the patient completes to provide the patient information. Although shown as a stand-alone application, it should be appreciated that a user interface in accordance with some embodiments may be implemented using a web browser interface such that the patient information may be provided on any device connected to the Internet.

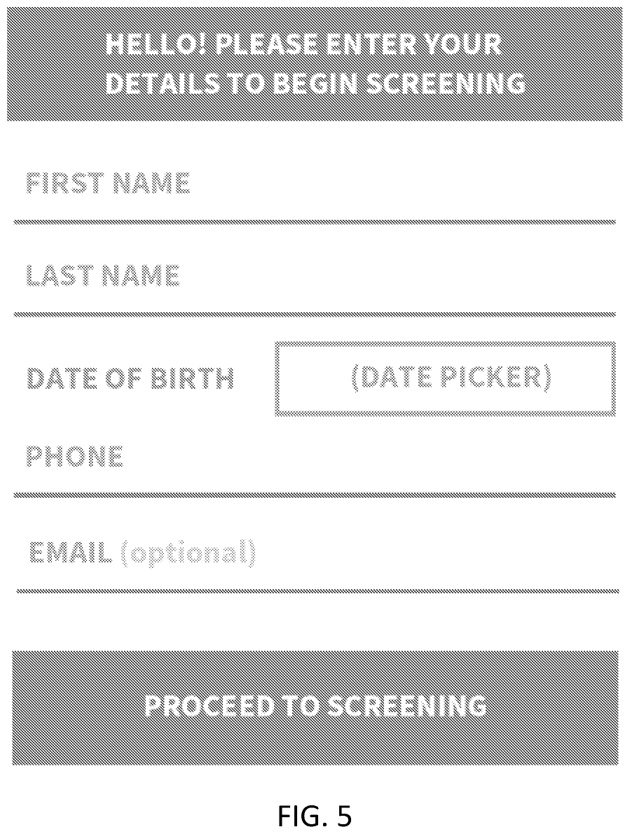

FIG. 5 illustrates a patient demographics portion of a user interface in which a patient is prompted to enter demographic information including, but not limited to, name, date of birth and contact information. In some embodiments, the patient demographic information is used to associate the patient with a unique identifier stored by the system to map the patient information entered into the questionnaire to particular patient account and/or electronic medical record.

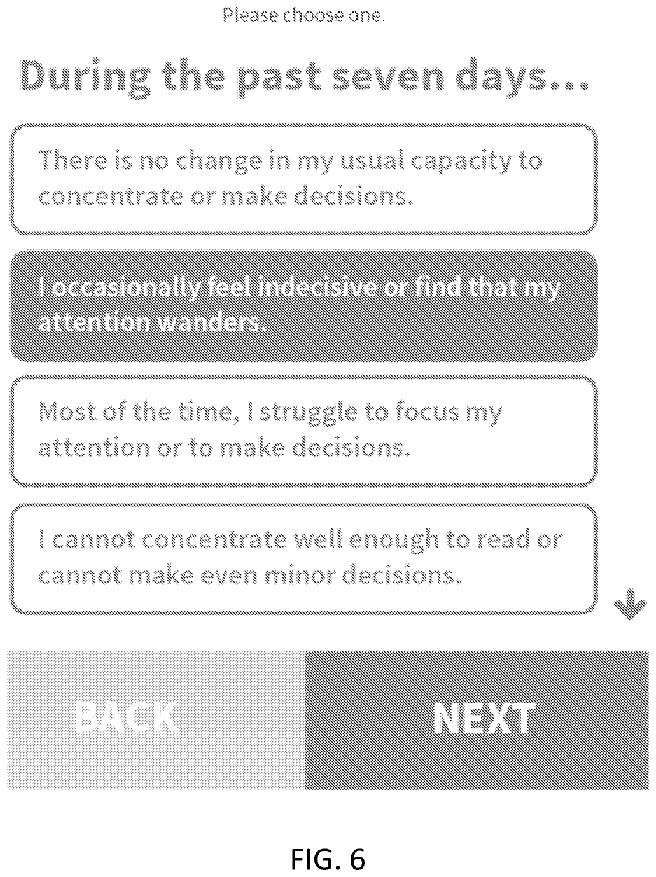

FIG. 6 illustrates a portion of a user interface that presents questions in a questionnaire to a patient for completion. As shown, each question in the questionnaire may be presented as a multiple choice question that enables the patient to select one of the available options to provide the patient information. In other implementations, the questionnaire may be designed to prompt the patient to rate particular symptoms or feelings on a scale (e.g., 1-4) for at least some of the questions to provide the patient information, or questions in the questionnaire may be formulated in any other suitable way.

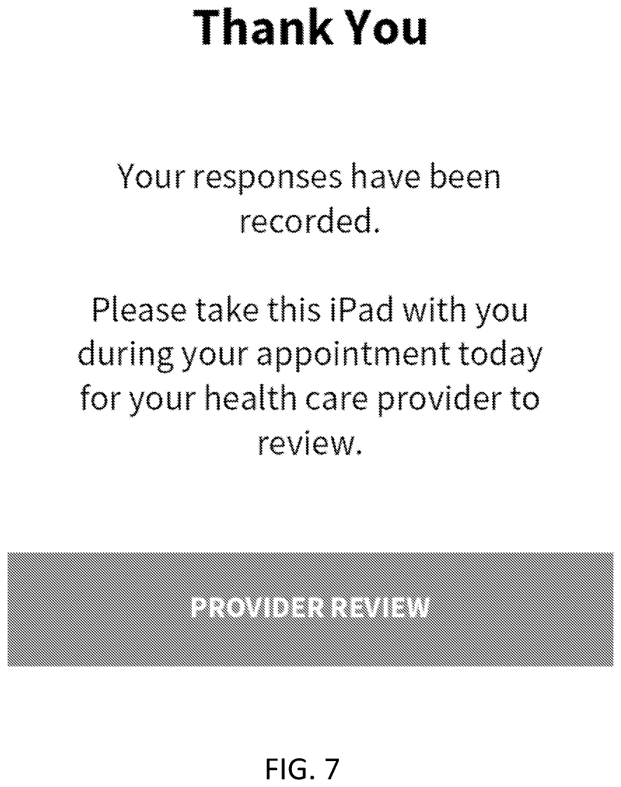

FIG. 7 illustrates a completion screen portion of a user interface that may be shown to the patient following completion of the questions in the questionnaire, an example of which is shown in FIG. 6. As shown, the completion screen portion of the user interface may include instructions informing the patient to provide the portable electronic device to their healthcare provider to enable the healthcare provider to review the results of the treatment outcome prediction based on their provided patient information.

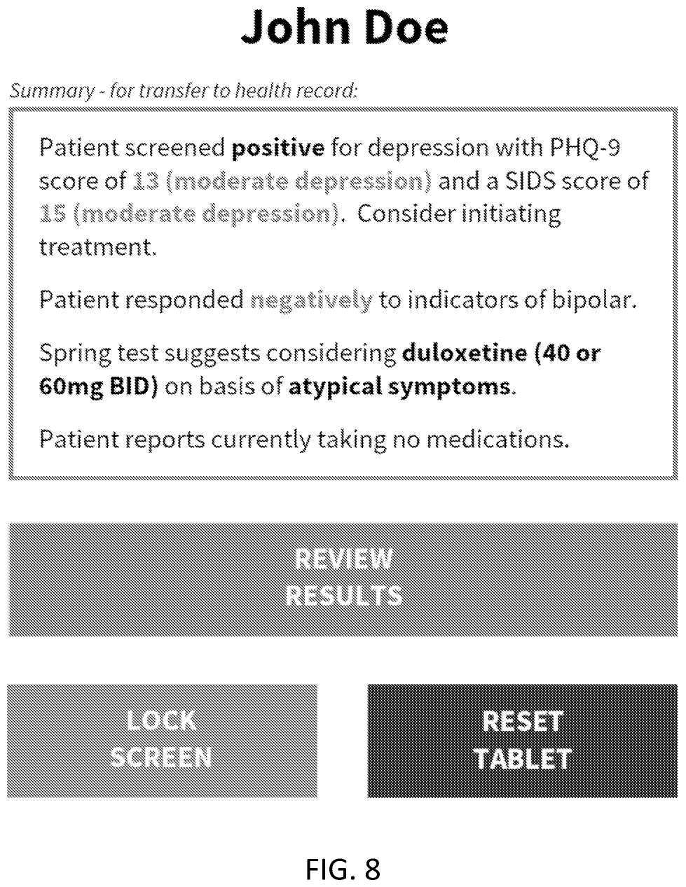

FIG. 8 illustrates a summary report portion of the user interface that may provide a healthcare provider with an overview of the result of processing the provided patient information by the trained statistical model in accordance with some embodiments. As shown, the summary report may display information about the patient's medical condition (e.g., estimated severity of depression), symptom-specific information, recommended treatment information and medical history information. It should be appreciated that the summary report may include additional or fewer types of information and embodiments are not limited in this respect. As shown, at least some of the information displayed in the summary report may be automatically transferred to an electronic health record of the patient.

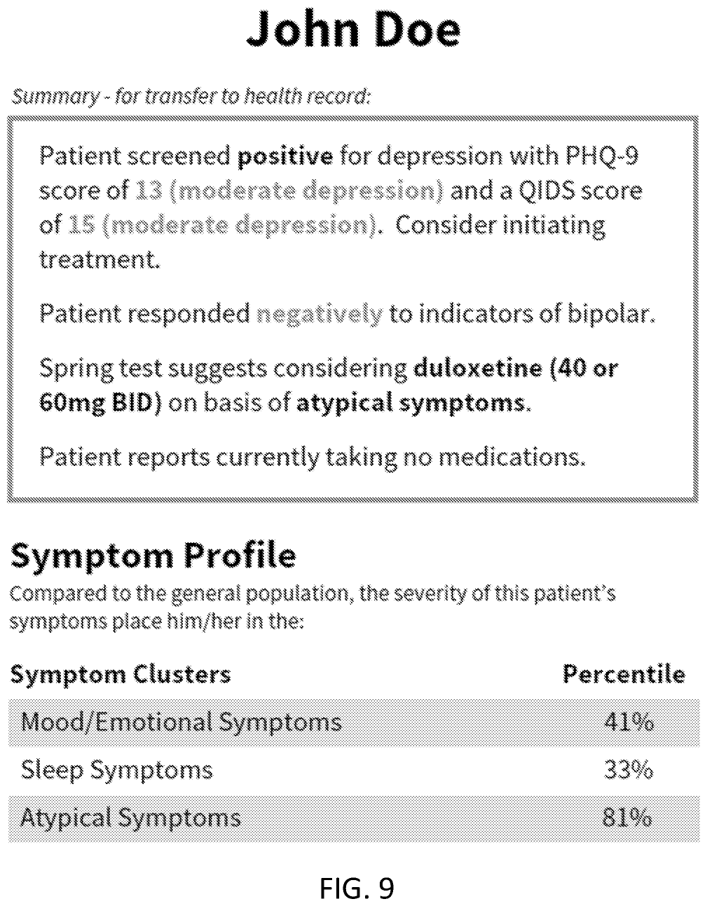

FIG. 9 illustrates another summary report portion of the user interface that provides information on how the trained model has classified the symptoms of the patient. As described in more detail below, some embodiments apply a symptom clustering technique that determines, based, at least in part, on the patient information provided by the patient, a likelihood that the patient is associated with one of a plurality of symptom cluster profiles.

The inventors have recognized and appreciated that certain types of treatments are more effective for patients that share a common set of symptoms. In some embodiments, the likelihood of a patient being associated with each of three symptom clusters is determined, where the three symptom clusters are “mood/emotional symptoms” often associated generally with depression, “sleep symptoms” indicating that the patient has difficulty sleeping, and “atypical symptoms,” for example associated with cognitive or speech difficulties. As shown, the summary report may display a probability that the patient exhibits a symptom profile that places the patient within each of the symptom clusters based on an analysis of the provided patient information using a trained clustering model. In the example provided, the patient is most closely associated with the “atypical symptoms” symptom cluster based on the patient information provided. Although three symptom cluster profiles are shown, it should be appreciated that fewer or additional symptom cluster profiles may be used in some embodiments.

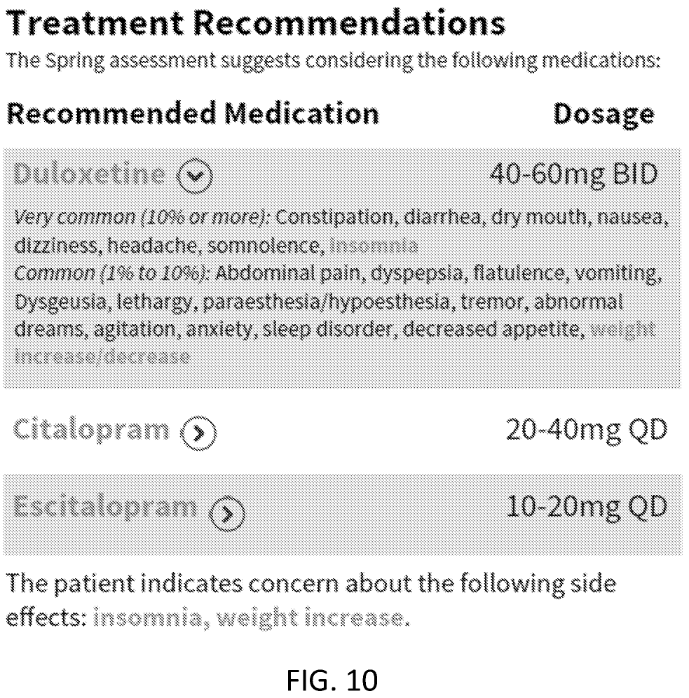

FIG. 10 illustrates a treatment recommendation portion of the user interface that displays recommended medications and dosages for the patient based on an analysis of the patient information by the trained statistical model. As shown, the treatment recommendation portion may include information about the recommended medications including side effects for the medications to enable the healthcare provider to make an informed treatment determination.

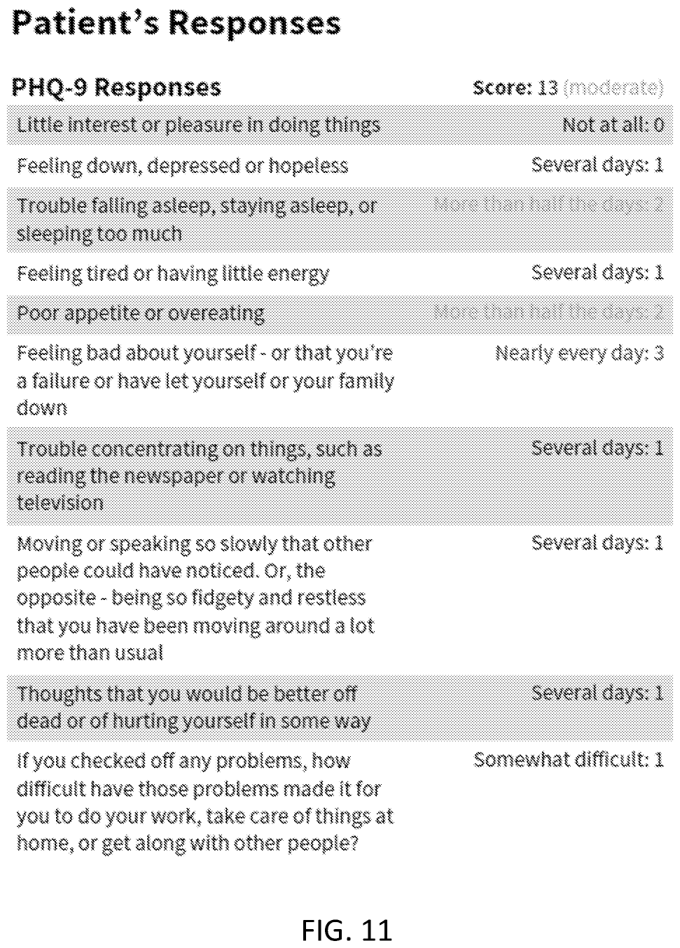

FIG. 11 illustrates a patient response summary portion of the user interface that enables a healthcare provider to drill down into individual patient responses, which enables the healthcare provider to further assess whether particular medications are appropriate for treating the patient and/or to ask the patient follow-up questions for patient responses to particular questions. In the example shown, the patient was associated with a Patient Health Questionnaire (PHQ-9) score of 13, and the patient response summary shows the breakdown of the individual questions that resulted in that particular score. By presenting the information in this way, the healthcare provider may be able to focus on the most severe symptoms or combination of symptoms when determining the best treatment course for the patient.

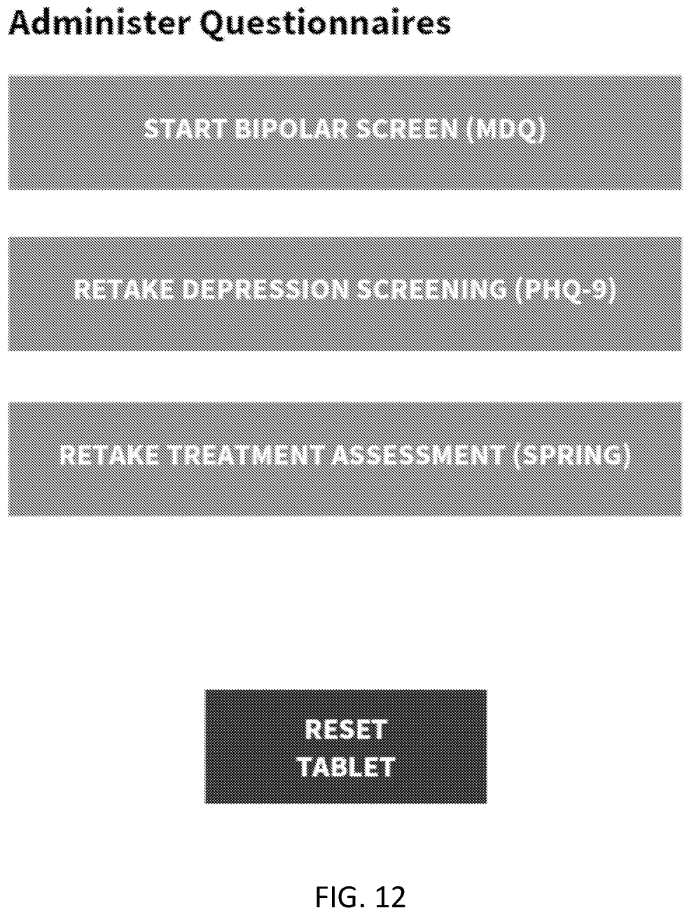

Some embodiments provide a user interface that enables a user to select from among multiple questionnaires for providing patient information. FIG. 12 illustrates a questionnaire selection portion of the user interface that enables a patient to select a questionnaire to complete. As shown, the questionnaire selection portion may displayed to the patient following completion of the depression screening questionnaire described above to enable the patient to answer the questions in the questionnaire again and/or to complete a different questionnaire.

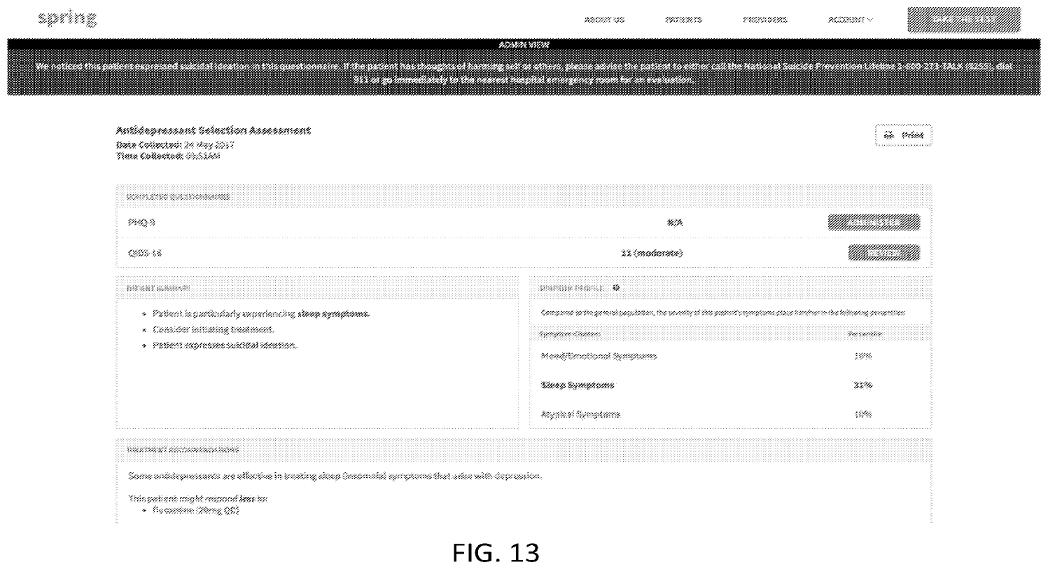

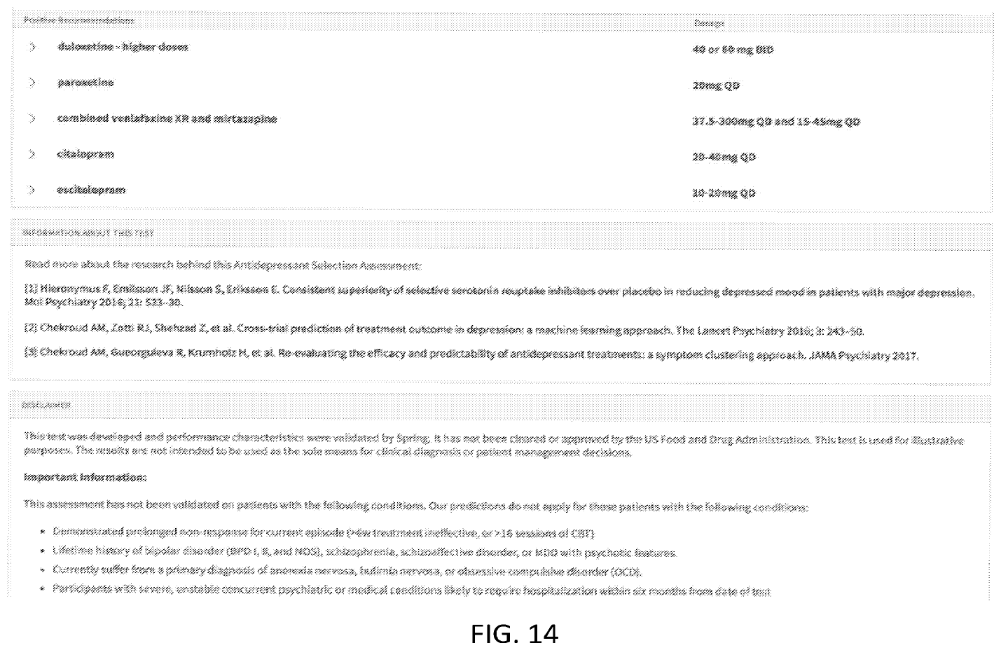

FIG. 13 shows an example of presenting a summary report generated as output of a trained statistical model using a web-browser user interface in accordance with some embodiments. As shown, the user interface shown in FIG. 13 combines several of the portions of the user interface shown in FIGS. 4-12 in the stand-alone application implementation. For example, the user interface shown in FIG. 13 includes a questionnaire selection portion, a patient summary portion that provides a healthcare provider with an overview of the patient's symptoms based on processing the provided patient information by a trained statistical model, a symptom profile portion that displays a likelihood that the patient is associated with one of a plurality of symptom cluster profiles, and a treatment recommendation portion. FIG. 14 shows additional information that may be presented on the web browser implemented user interface of FIG. 13 including information about recommended medications and other information about the patient assessment tool developed in accordance with some embodiments.

### Example Model Development

In one implementation in accordance with some embodiments, a 25-item questionnaire was developed to ask patients about their medical/psychiatric background and current symptom profile. The information received from the questionnaire was then provided as input to a statistical model that employed a machine learning technique to predict whether a depressed patient will reach clinical remission with a 12-week course of a specific antidepressant. The model comprised a plurality of decision trees (e.g., 370 decision trees) with the output of the decision trees being aggregated using a weighting function. The model was rigorously validated in two independent clinical trials and outperformed all currently available predictive tools.

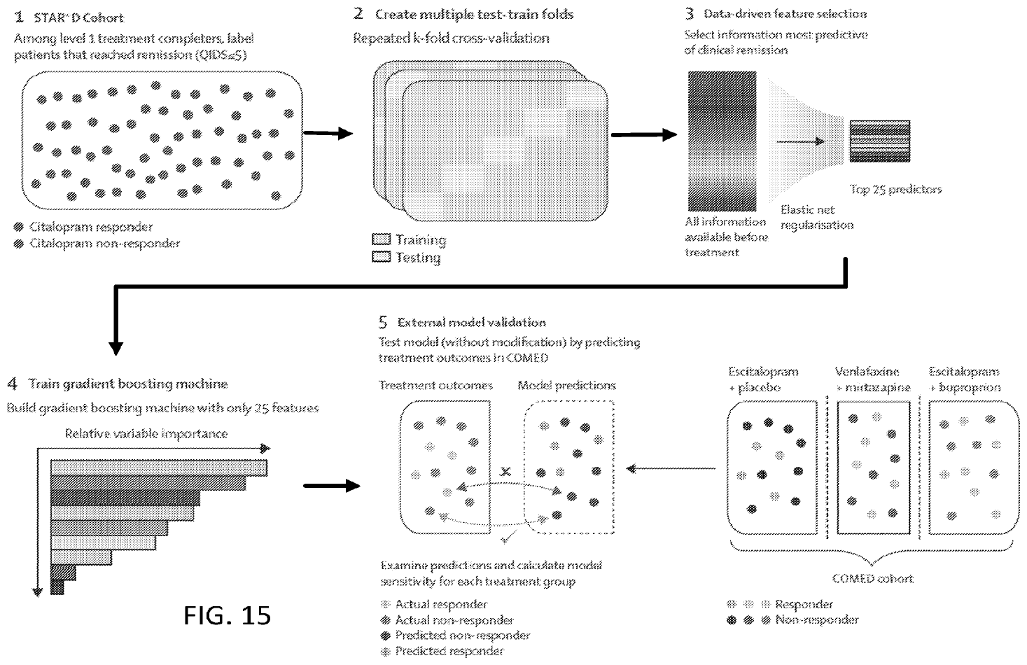

FIG. 15 illustrates a flow chart of a process used to develop and validate the statistical model using data obtained from two large scale multi-center clinical trials of major depressive disorder (Sequenced Treatment Alternatives to Relieve Depression (STAR*D), clinicaltrials.gov number NCT00021528 and Combining Medications to Enhance Depression outcomes (CO-MED) clinicaltrials.gov number NCT00590863) from the National Institutes of Mental Health (NIMH). The STAR*D clinical trial is the largest prospective, randomized controlled study of outpatients with major depressive disorder. Patients were recruited from primary and psychiatric care settings from June, 2001, to April, 2004. Eligible participants were treatment-seeking outpatients, with a primary clinical Diagnostic and Statistical Manual of Mental Disorders Version, Fourth Edition (DSM-IV) diagnosis of non-psychotic major depressive disorder, a score of at least 14 on the 17-item Hamilton Depression Rating Scale (HAM-D), and aged 18-75 years. Because the focus of the statistical model in this example was to predict initial antidepressant response, data from the first treatment stage—a 12-week course of citalopram, a commonly used selective serotonin reuptake inhibitor (SSRI) antidepressant was used.

The CO-MED clinical trial was a single-blind, randomized, placebo-controlled trial comparing efficacy of medication combinations in the treatment of major depressive disorder. Briefly, 665 outpatients were enrolled between March, 2008, and February, 2009, across six primary care sites and nine psychiatric care sites. Eligible patients were aged 18-75 years, had a primary DSM-IV-based diagnosis of non-psychotic major depressive disorder, had recurrent or chronic depression (current episode ≥2 years), and had a score of at least 16 on the 17-item HAM-D rating scale. Exclusion criteria included all patients who had comorbid psychotic illness or bipolar disorder, or who needed admission to hospital. Patients were randomly allocated (1:1:1) to one of the following three groups: escitalopram plus placebo (monotherapy); escitalopram plus bupropion; or venlafaxine plus mirtazapine.

As shown in step 1 of FIG. 15, a subset of all patients in the STAR*D cohort were selected to create a first dataset used to determine the features for the statistical model and to train the model. In particular, patients determined as level 1 treatment completers according to whether they reached remission (Quick Inventory of Depressive Symptomatology (QIDS) score ≤5) or not were selected as the patients to include in the first dataset used for training the model. A complete cases approach was used by including only patients without missing observations. Although patients in both trials were encouraged to visit the clinic every two weeks, most patients did not attend every appointment. The analyses in this example focused on patients for whom a severity score was recorded after 12 or more weeks of treatment. Of the original 4041 patients in STAR*D, 12 had no outcome data, and 2044 completed fewer than 12 weeks of treatment, resulting in 1985 patients who had complete case data. For CO-MED, of the original 665 patients, two had no outcome data, and 187 did not complete 12 weeks, resulting in 476 patients who had complete case data. Patients with missing baseline data (36 patients in STAR*D and 51 patients in CO-MED) were excluded. The statistical model in this example was trained using data from the 1949 patients from STAR*D who had complete case data and no missing baseline data. 425 patients were included in a second dataset for external validation in CO-MED (escitalopram-placebo 151; bupropion-escitalopram 134; venlafaxine-mirtazapine 140). Baseline depressive severity was similar for the final completer sample (mean Quick Inventory of Depressive Symptomatology [QIDS] severity 15.1, range 2-27, Interquartile Range (IQR) 12-18) and those excluded for missing outcome data (mean 15.9, range 2-27, IQR 13-19).

Outcome measurements for the patients were assessed according to the 16-item self-report QIDS (QIDS-SR16) with a focus on the key clinical target of clinical remission (final score ≤5 in the 16-item QIDS-SR16), since it is associated with better function and a better prognosis than response without remission. Since visit weeks differed between the two trials, for STAR*D, final scores were determined as the last available measurement at either 12 or 14 weeks; for CO-MED, final scores were determined at 12 or 16 weeks. Prediction of other clinical outcomes of interest (e.g., percentage symptom change) would be possible using similar techniques.

As shown in step 2 of the analysis pipeline shown in FIG. 15, all models were constructed and examined with repeated ten-fold cross-validation (ten repeats), which partitions the original sample into ten disjoint subsets, uses nine of those subsets in the model training process, and then makes predictions about the remaining subset not used for training. To avoid opportune data splits, model performance metrics were averaged across test folds. For external validation, the trained statistical model built using the first dataset from the STAR*D cohort was applied without modification to predict treatment outcomes in each CO-MED treatment group separately.

Because the STAR*D and CO-MED clinical trials were completed many years ago, the clinical outcome for each patient (i.e., whether they reached clinical remission or not) was known. In statistical terms this means that the dataset was “labelled” with a particular outcome. All readily available sources of information that overlapped for patients in both STAR*D and CO-MED trials were extracted from the trial records that were available about each patient at baseline (i.e., before treatment commenced). Information included various sociodemographic features, DSM-IV-based diagnostic items, depressive severity checklists (e.g., QIDS-SR and HAM-D), eating disorder diagnoses, whether the patient had previously taken specific antidepressant drugs, the number and age of onset of previous major depressive episodes, and the first 100 items of the psychiatric diagnostic symptom questionnaire.

A key challenge for prediction is to identify which features or variables to use in the statistical model. Machine-learning techniques seek to identify predictive variables (also called “features” herein). In the present context, this problem is particularly salient: although models could benefit from more variables, the model loses utility as more and more questions are asked of the patient (as implementation becomes more effortful). A typical solution to this problem is to use a stepwise feature selection procedure, but this approach is slow and prone to over-fitting. Step 3 of the analysis pipeline shown in FIG. 15 schematically illustrates a process for identifying variables that are most predictive of treatment outcome using a data-driven selection process in accordance with some embodiments.

In this example, 164 predictors were modeled simultaneously with an elastic net regularization method that avoids issues of correlated predictors and reduces the risk of over-fitting. The method had two primary effects: coefficients of correlated predictors are shrunk towards each other, and uninformative features are removed from the model. Mathematically, the method linearly combines the β weight penalties of the lasso (l₁-norm) and ridge (l₂-norm) regressions. As such, the elastic net solves the following problem:

$${min}_{{β}_{0} , β} \frac{1}{N} {\sum }_{i = 1}^{N} {w}_{i} l ( {y}_{i} , {β}_{0} + {β}^{T} {x}_{i} ) + λ [ ( 1 - α ) {‖ β ‖}_{2}^{2} / 2 + α {‖ β ‖}_{1} ] ,$$

over a grid of values of λ covering the entire range. Here l(y,η) is the negative log-likelihood contribution for observation i; e.g., for the Gaussian case l(y,η) is ½(y-η)². The elastic-net penalty is controlled by α, where the lasso is α=1 and ridge is α=0. The tuning parameter λ controls the overall strength of the penalty. The elastic net approach maintains model parsimony by explicitly penalizing overfitting, and yields stable and sparse models that are robust to multicollinearity among features. Each variable was centered and then scaled (resulting in a mean of 0 and standard deviation of 1) before entry into the elastic net model to account for difference in variable types and ranges.

The elastic net model was used to select the 25 most predictive variables from those available using the STAR*D training sample. The concept of nuisance covariates does not apply since all information extracted from the trial was included in the model (that is, all information was of interest). This two-step procedure of preselecting variables before final model building ensured that the final predictive model would need only 25 variables. The top 25 most predictive variables were selected in this example to develop a questionnaire that was clinically manageable, though it should be appreciated that any other suitable number of variables may alternatively be selected for inclusion in the statistical model.

Following the variable selection stage, the predictive features were used to train a statistical model to predict treatment outcomes for a particular antidepressant as schematically illustrated in step 4 of the analysis pipeline shown in FIG. 15. A combination of supervised machine learning methods were used to predict clinical outcomes—in particular whether each patient would reach clinical remission for a 12-week course of a specific antidepressant treatment. In this example, the model was trained using a first dataset of labeled data from the first clinical trial, STAR*D. The 25 predictive variables selected using the elastic net model were used to train a statistical model to predict clinical remission. The machine learning technique used in this implementation was a gradient boosting machine, with the model comprising a weighted combination of individual decision trees. Rather than fitting one strong model to a dataset, a gradient boosting machine is built by combining several weakly predictive models to relate the predictors and outcome. When each successive model is fit, the model focuses on the data that previous models failed to predict. A tree-based ensemble of 370 individual decision trees was fit to the top 25 predictors identified by the elastic net model.

In this example, the statistical model was developed to detect patients for whom citalopram was beneficial (rather than predicting non-responders). Optimum tuning parameters were selected during cross-validation through an area under the receiver-operating curve (ROC)-maximization process (comparing true positives to false positives). The best performing model in the training dataset was used to generate predictions in the independent validation set. The significance of the model's accuracy with a one-tailed binomial test of model accuracy relative to the bigger class proportion (null-information rate) was determined. Other relevant descriptions of model discrimination were also measured including sensitivity, specificity, and area under curve (AUC)—at each stage.

The statistical model developed using data from the first clinical trial (STAR*D) was then validated prospectively with data from a second, independent clinical trial (CO-MED) as schematically illustrated in step 5 of the analysis pipeline shown in FIG. 15. The model was applied to this data without modification. The second trial included three different treatment randomization arms. The model predicted outcomes with above chance accuracy in the two treatment arms where patients took treatments that were primarily serotonergic in nature (i.e., SSRI based). Accordingly, the model was also validated to perform statistically above chance when predicting outcomes for Escitalopram (Lexapro) monotherapy, and also a combination regimen of Escitalopram plus Bupropion (Lexapro plus Wellbutrin). The statistical model—trained on a SSRI treatment—did not accurately predict treatment outcomes for a serotonin-norepinephrine reuptake inhibitor (SNRI)-based treatment regime (Venlafaxine+Mirtazapine), suggesting that the model may have learned something that is specific to the underlying pharmacotherapeutic mechanism of serotonergic interventions (like Citalopram and/or Escitalopram).

Twenty-five predictors of remission or non-remission according to ranked absolute beta weights in the elastic net model were selected. The top three predictors of non-remission were baseline QIDS-SR depression severity, feeling restless during the past 7 days (QIDS-SR psychomotor agitation), and reduced energy level during the past 7 days (QIDS-SR energy and fatiguability). The top three predictors of remission were identified as currently being employed, total years of education, and loss of insight into one's depressive condition (HAM-D loss of insight).

A statistical (machine-learning) model was constructed with the selected set of 25 variables, as discussed above. Patient information comprising responses to 25 questions were provided as input to the statistical model which predicted a likelihood that the patient will respond to Citalopram with a 12-week course of the antidepressant. The model was rigorously validated using repeated cross-validation, and performed with significantly above chance accuracy. Specifically, the model achieved an average AUC of 0.700 (SD 0.036), suggesting sufficient predictive signal in the 25 questions selected by the elastic net. The majority class was non-remission, comprising 51.3% of patients (null information rate). Overall, the model's predictions had significant accuracy in predicting outcome in STAR*D patients (accuracy 64.6% [SD 3.2]; p<9.8×10⁻³³). The model prospectively identified 62.8% (SD 5.1) of patients who eventually reached remission (i.e., sensitivity), and 66.2% (SD 4.6) of non-remitters (i.e., specificity). Correspondingly, the model had a positive predictive value (PPV) of 64.0% (SD 3.5), and a negative predictive value (NPV) of 65.3% (SD 3.3).

For comparison, a group of practicing psychiatrists were asked to predict outcomes for a subset of patients from the first trial using information available at baseline, and the psychiatrists were found to have below chance accuracy. The ability of the statistical model developed in accordance with some embodiments to predict treatment outcomes with better accuracy than conventional non-computer-implemented prediction techniques demonstrates the technical improvement of some embodiments with respect to the underlying treatment outcome prediction process.

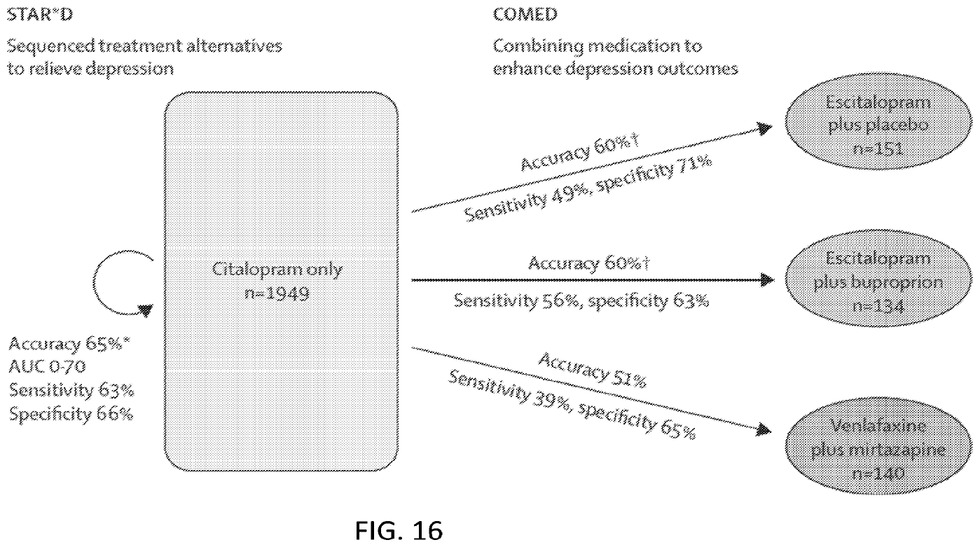

To confirm the model's external generalizability, the 25-item model from the STAR*D citalopram completers (without modification) was applied to patients in the three CO-MED treatment groups separately. FIG. 16 shows the pattern of cross-trial model performance. The arrows in FIG. 15 indicate where a model was trained (arrow origin) and tested (arrow head), with *p<0.001, and †p<0.05. The model showed significant predictive performance in both the escitalopram-placebo group (79 remissions; accuracy 59.6%, 95% CI 51.3-67.5, p=0.043; PPV 65.0%, NPV 56.0%), and escitalopram-bupropion group (66 remissions; accuracy 59.7%, 50.9-68.1, p=0.023; PPV 59.7%, NPV 59.7%), but not the venlafaxine-mirtazapine group (72 remissions; accuracy 51.4%, 42.8-60.0; p=0.53; PPV 53.9%, NPV 50.0%). These differences in the predictability of treatment response occurred even though the overall treatment efficacies were similar in the three groups (escitalopram-placebo 52.3%, escitalopram-bupropion 49.3%, venlafaxine-mirtazapine 51.4%). For completeness, the same general modelling pipeline was applied to each CO-MED treatment group separately.

A parallel approach was used to predict the final QIDS-SR scores for STAR*D patients directly, rather than by use of remission versus non-remission status. A substantial overlap was identified in the top 25 variables: the regression and classification models share 16 of their top 25 predictors, and eight of the top ten remain the same. In this format, the statistical model trained on the STAR*D data explained 17.5% of the variance in final QIDS-SR scores (root mean square error [RMSE] 4.54 [SD 0.20], R²=0.175 [SD 0.052]).

Including as one of the 25 variables the total QIDS-SR score measured at two weeks after baseline substantially improved all cross-validated performance measures in STAR*D completers relative to the baseline-only model

In this example, a model was developed to predict symptomatic remission after taking citalopram, a common antidepressant, with clinical rating data. The model performance was similar to that of calculators of disease risk, recurrence, or treatment response in various areas of medicine, including oncology and cardiovascular disease. In the context of depression, the model performed comparably to the best available biomarker—an EEG-based index13 but is less expensive, easier to implement, and validated in large internal and external clinical trial samples. The model was optimized to detect future responders, and improved substantially if a self-reported measure of overall depressive severity after two weeks of treatment was included in the model, indicating the possible usefulness of a two-week prediction update that may be implemented in some embodiments.

A personalized medicine approach to pharmacotherapy holds promise in treatment of depression, a highly heterogeneous illness for which no single treatment is universally effective, and for which many patients undergo several treatments before an appropriate regimen is identified. From large-scale clinical trials (including STAR*D and CO-MED), at a population level, about 30% of patients achieve symptomatic remission for a given treatment and episode. However, personalized medicine shifts focus away from population remission rates and general drug efficacy, and tries to identify which 30% of patients are the best candidates for a specific drug. As an example, although remission rates for all drug treatments were similar (48-52%), the ability to prospectively predict treatment outcome was not (51-65%).

The success of these models may depend, at least in part, on their ability to generalize. In this implementation, all variable selection and model building occurred during a repeated ten-fold cross-validation procedure. Additionally, it was examined how the model trained on a large citalopram cohort would perform in other clinical trial cohorts with other treatment protocols, with differing recruitment criteria and distributions of symptoms. The external validation analysis showed that a citalopram model trained in the STAR*D cohort accurately predicted outcomes for the escitalopram treatment group of CO-MED. The model also showed significant accuracy in the escitalopram-bupropion group, but not in a combination SNRI group (venlafaxine-mirtazapine). This result shows that a statistical model trained in accordance with some embodiments can successfully generalize to a completely independent sample, and has some degree of treatment specificity. The fact that the model failed to accurately predict response to the venlafaxine-mirtazapine group suggests that the model was not simply predicting a generic treatment response profile, nor was it predicting regression to the mean (or performance would have been equivalent for all three CO-MED groups). The use of wholly independent validation cohorts also showed that, although fewer predictors might still confer comparable model performance in the STAR*D cohort, these smaller models did not generalize to the escitalopram group of an independent clinical trial, highlighting the importance of external validation.

It is generally accepted that statistical (and biomarker) models must show above chance performance to be useful. In the STAR*D analyses described herein, an accuracy of 53.13% would have statistically outperformed the chance accuracy of 51.3% with this sample size, by conventional standards (p<0.05). The model described herein achieved an accuracy of 64.6%, surpassing this benchmark substantially. By contrast, clinical prediction regarding which patients will respond to which treatment is typically poor.

Some embodiments are directed to using symptom clustering to identify structure with a clinical rating scale. Medical conditions such as depression are often assessed using questionnaires that include a diverse set of questions. For example, a questionnaire for a depression rating scale may include questions related to “waking up at night,” “low libido,” and “difficulty concentrating. The inventors have recognized and appreciated that by analyzing patient responses to the different types of questions in a clinical rating scale questionnaire using a clustering technique, patients may be associated, based on their symptom profile to one of a plurality of symptom cluster groups, each of which may be associated with medications that are more effective in treating the symptoms of that group. In some embodiments, a symptom clustering technique may be used in combination with the predictive statistical model described above. However, in other embodiments, the symptom clustering technique may be used without predictive modeling.

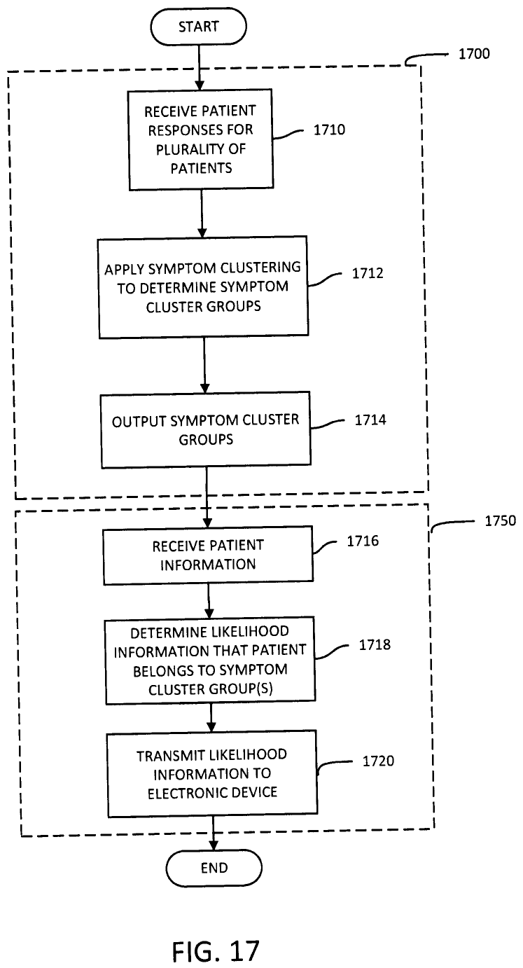

FIG. 17 illustrates a flowchart of a process for generating 1700 and using 1750 a symptom cluster model in accordance with some embodiments. As shown, the process of generating a symptom cluster model may comprise act 1710 in which patient responses to questions on a clinical rating scale questionnaire for a plurality of patients are received. The process then proceeds to act 1712, where the received patient responses are processed using a symptom clustering technique to determine a plurality of symptom cluster groups. In the example described below a hierarchical clustering techniques is applied to a large dataset of patient information to determine a plurality of symptom cluster groups for depression including a sleep symptom group, a core emotional symptom group, and an atypical symptom group. It should be appreciated that other symptom clustering techniques may be additionally or alternatively used. Following determination of the plurality of symptom cluster groups, the process proceeds to act 1714 where an indication of the symptom cluster groups is output. In some embodiments, outputting an indication of the symptom cluster groups comprises outputting a trained model that can then be used for classification of patients based on their symptom profile.

An example of using a symptom cluster model that has been generated in accordance with process 1700 is illustrated as process 1750 in FIG. 17. In act 1716 patient information for a patient is received. The patient information includes patient responses to questions in a questionnaire. The process then proceeds to act 1718, where the received patient information is processed in accordance with the symptom cluster model to determine a likelihood that the patient is associated with one or more of the symptom cluster groups in the system cluster model based on the patient's symptom profile as determined from the received patient information. The process then proceeds to act 1720, where the likelihood information is transmitted to an electronic device for display, for example, to a healthcare provider to facilitate a treatment decision. An example of displaying the likelihood information as a portion of a user interface of an electronic device is shown in FIG. 9. As shown, a likelihood that a patient is associated with each of three symptom cluster groups is displayed on the user interface. The likelihood information may alternatively be provided to a healthcare provider in any suitable way.

### Example Symptom Clustering Implementation

In this example, a symptom clustering technique is used to determine symptom cluster groups to develop a symptom cluster model and using the symptom cluster model to provide treatment outcome predictions based, at least in part, on which of a plurality of symptom cluster groups a patient was associated with based on provided patient information. The symptom cluster groups identified in this example include sleep (insomnia), emotional, and atypical, though it should be appreciated that other symptom cluster groups may alternatively be used.

Meta-analyses and factor analytic studies of large populations with depression indicate that the symptoms of major depressive disorder are organized into two to five clusters depending on the checklist used. Nevertheless, clinical trials of patients with depression nearly always report total symptom severity scores as their primary outcome measures. These studies also frequently report the proportion of patients whose total symptom severity falls below a certain threshold and thus achieve clinical response or remission.

Heterogeneity among depressive symptoms may impede the evaluation of treatments for depression. For example, treatment efficacy for one group of symptoms may be masked by a lack of efficacy for other symptoms, potentially explaining mixed results from large comparative efficacy meta-analyses. For example, SSRI-based antidepressants are generally effective in reducing low mood relative to other symptoms. However, evaluating outcomes on an individual symptom level may be cumbersome since clinicians would need to remember treatment guidelines specific to each symptom. Although symptoms might be grouped based on clinical experience (e.g., “melancholic depression”) or the use of rating subscales (e.g., Hamilton Rating Scale for Depression), novel associations might be overlooked by this process.

Statistical methods enable one to categorize depressive symptoms into subcomponents. For example, one study showed that nortriptyline hydrochloride is more effective than escitalopram in treating a neurovegetative symptom dimension, but escitalopram was more effective in treating mood and cognitive symptom dimensions. However, traditional statistical approaches have shortcomings. Factor analyses, for example, may generate complicated combinations of symptoms within particular dimensions. These analyses also may be susceptible to experimenter bias since one often has to choose the desired number of clusters or components in the data, as in k-means clustering. By contrast, hierarchical clustering as used herein is an easy-to-visualize, deterministic method in which each symptom is assigned to a single cluster (i.e., not loading across multiple clusters) without pre-specifying the desired number of clusters.

In this example, the efficacy and predictability of antidepressant therapies was explored in treating specific groups of symptoms. Hierarchical clustering, which is an unsupervised machine-learning technique was used to establish a data-driven grouping of baseline symptoms. The clustering method was applied to patients from a large multisite trial of depression (STAR*D) and a replication sample from an independent clinical trial with similar inclusion criteria (CO-MED). Next, treatment outcomes for nine archival clinical trials were analyzed according to the severity of each symptom cluster (rather than total severity) to determine whether symptom clusters are equally responsive to antidepressant treatments and whether certain drugs and doses are more effective than others. Finally, supervised machine learning using a trained statistical model, as described above, was used to predict outcomes specific to each cluster of symptoms since there may be good clinical or biological indicators of changes in some symptoms that do not correlate strongly with changes in other features of depression.

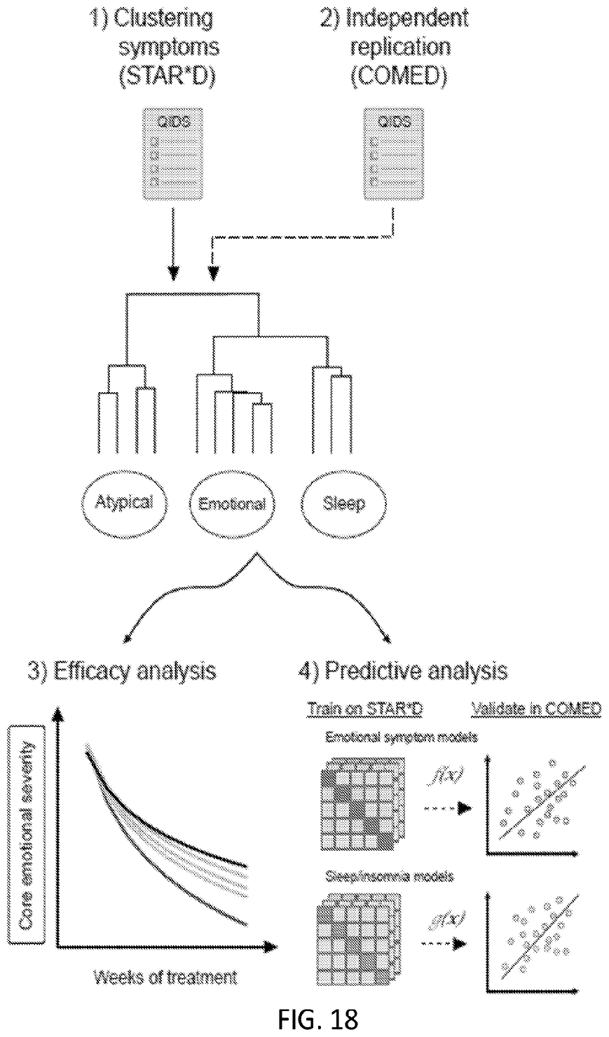

FIG. 18 schematically provides an overview of a process for developing a model to predict treatment outcomes based, at least in part, on symptom clustering of patient information, in accordance with some embodiments. As shown, in step 1, symptoms on the QIDS checklist were clustered based on the STAR*D cohort. In step 2, a clustering analysis was replicated on an independent sample (data from the CO-MED trial). In step 3, trial outcomes were reanalyzed for 9 clinical trials according to each symptom cluster. In step 4, outcomes specific to each symptom cluster were predicted using machine learning, by training statistical models STAR*D (using cross-validation), and then validating the models in CO-MED as described above.

Rating scales in depression include a diverse range of symptoms. A data-driven approach was applied to identify groups of symptoms within depression rating scales. Higher scores on the rating scales indicate more severe symptoms. Hierarchical clustering shows structure in data without making assumptions about the number of clusters that are present in the data and gives a deterministic solution.

Agglomerative (bottom-up) hierarchical clustering was applied to the QIDS-SR checklist completed at baseline in STAR*D by 4017 patients and the analysis was replicated using baseline QIDS-SR data from CO-MED (n=640) and the baseline HAM-D scale that was also collected on 4039 patients in STAR*D. Multiple sensitivity analyses were conducted using alternative approaches. With this procedure, values relative to the merge points of their subgroups are considered candidates for natural clusters. Symptom similarity was defined by the Manhattan distance (also called “cityblock” distance). Hierarchical clustering was then conducted on this dissimilarity matrix. The resulting dendrogram was then pruned using the hybrid method, and the least restrictive minimum cluster size (minimum cluster size=1). A permutation-based test was used to ensure that the derived clusters were statistically reliable (e.g., permutation-based cut points had to be lower than or equal to the cut points determined through dynamic tree cutting), with 100 permutations.

For the HAM-D clustering, seven placebo-controlled phase 3 trials of duloxetine measured outcomes according to the HAM-D scale were used rather than the QIDS. In the STAR*D trial, a HAM-D checklist was also completed at baseline for 4039 patients (although not longitudinally). In order to conduct comparable symptom-cluster efficacy analyses of these additional datasets that used HAM-D longitudinally, a clustering analysis on the HAM-D checklist that was completed at baseline in STAR*D was used. As for the QIDS clustering, weight and appetite items were excluded. In addition, the HAM-D loss of insight item (e.g., “Denies being ill at all”) was also excluded from analysis as it can only be determined by a clinician and has no equivalent construct in the QIDS checklist. The hybrid dynamic tree cutting procedure was modified slightly, cutting at 80% of the range between the 5th percentile and the maximum of the joining heights on the dendrogram, to improve comparability of the cut points used across the two checklists. Nonetheless, the tree was still cut substantially higher than the minimum cut points identified through similarity profiling.

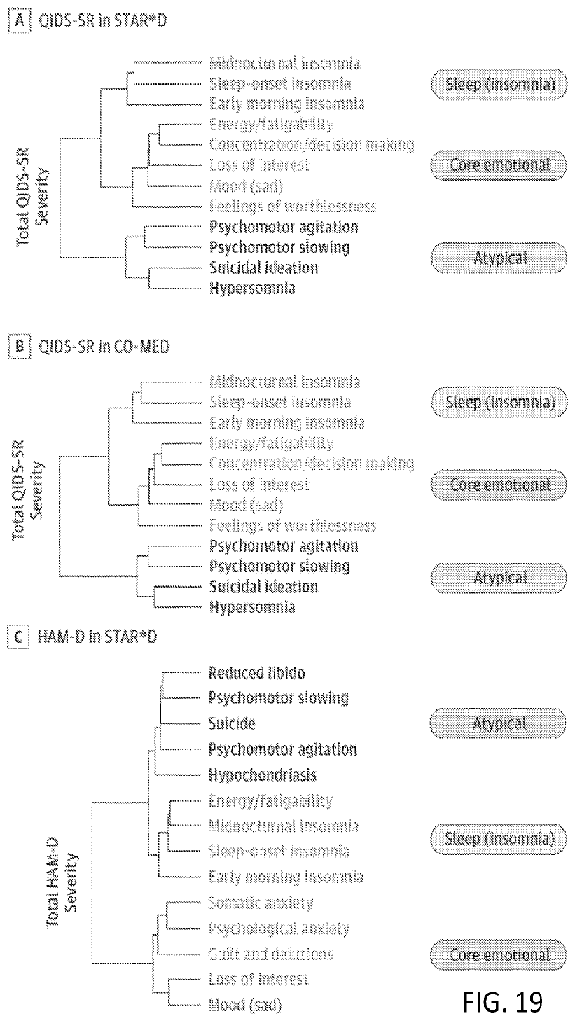

Results of the procedure for sequentially grouping symptoms according to the similarity of their responses across a patient cohort in accordance with some embodiments are shown in FIG. 19. With this procedure, groups of symptoms that merge at high values relative to the merge points of their subgroups are considered candidates for natural clusters. In FIGS. 18A and B, the Quick Inventory of Depressive Symptomatology-Self Report (QIDS-SR) checklist were used to identify an identical 3-cluster solution in both the STAR*D (n=4017) and CO-MED trials (n=640). As shown in FIG. 19C, a comparable symptom structure was also observed at baseline for STAR*D patients when measured according to the HAM-D rating scale. The names of the individual checklist items are shown according to their cluster assignment. Line lengths in the dendrogram reflect how similar items or clusters are to one another (shorter line length indicates greater similarity).

The full intent-to-treat samples in all trials was analyzed using linear mixed-effects regression models (STAR*D, 4041; CO-MED, 665; and other trials, 2515). The dependent measure was mean within-cluster severity: for each patient at each time point, the mean symptom severity was calculated within each cluster. Fixed effects included symptom cluster, time (log transformed weeks), treatment regimen, and all 2- and 3-way interaction effects. A separate random intercept and slope was included for each symptom cluster with unstructured variance-covariance of the random effects within subject based on improvements in the Schwarz-Bayesian information criterion. False-discovery rate-adjusted P values were used to determine statistical significance for post hoc comparisons by cluster and drug within each mixed-model analysis. One model was used to analyze QIDS-SR-based clusters across STAR*D and CO-MED, and another model was used to analyze HAM-D-based clusters for the 7 other placebo controlled trials. In the HAM-D model, the main effect of the trial was also included to control for potential systematic differences among trials. Preliminary analyses of the 4 duloxetine doses in each cluster indicated that 120-mg/d and 80-mg/d dosages were not significantly different from each other but differed from the lower doses and placebo. The 60-mg/d and 40-mg/d duloxetine dosages were similar to each other and nearly indistinguishable from placebo. The cohorts were grouped into high-dose duloxetine (80-120 mg/d) and low-dose duloxetine (40-60mg/d).

The statistical modeling pipeline shown in FIG. 15 was used to predict treatment outcomes specific to each symptom cluster using information available at baseline. As described above, 164 items, including demographics, medical and psychiatric histories, and specific symptom items were extracted and used as predictor variables. Penalized logistic regression (e.g., elastic net model) was then used to identify the 25 variables that best predicted each cluster separately. These variables were then used to train a statistical model (e.g., gradient boosting machines), resulting in a separate model for each symptom cluster, with each using 25 predictor variables. Predictability was measured as the percentage of variance explained in final cluster scores (i.e., R²) using 5 repeats of 10-fold cross-validation. The statistical significance of each model was assessed using a permutation test. The models were trained on patients with complete baseline data for whom a severity score was recorded after 12 or more weeks of treatment (n=1962) to ensure adequate treatment duration. To externally validate the predictive models, each model was applied without modification to predict final cluster scores in a second dataset of CO-MED treatment completers. Statistical significance was measured by a P value calculated for Pearson correlations between predicted outcomes and observed outcomes in each treatment group of CO-MED. For significance, permutation-based tests used an α level of 0.01, mixed-effects regressions used a false-discovery rate correction and then an α level of 0.05, and Pearson correlations used an α level of 0.05.

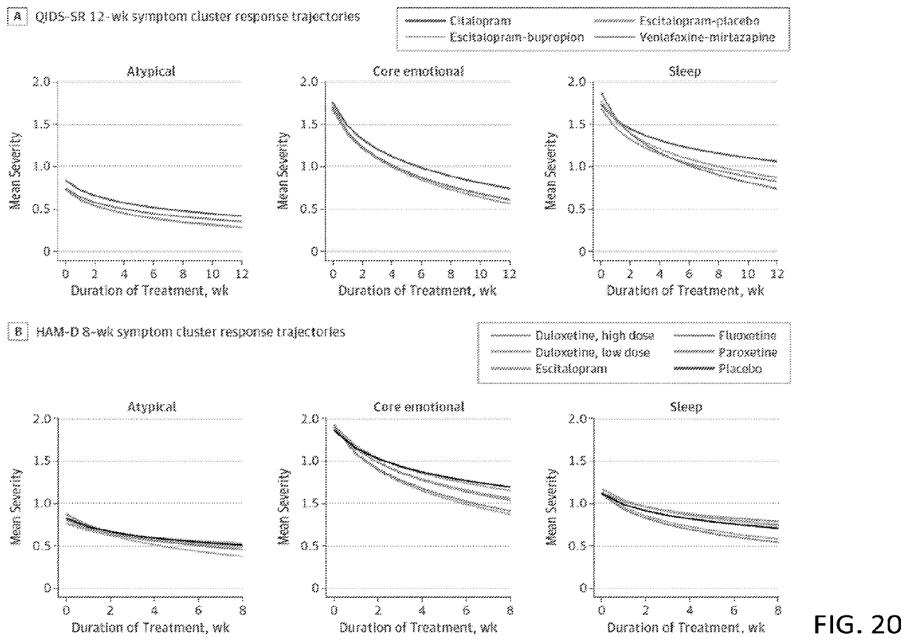

Treatment efficacy was measured according to the rate of symptom improvement over time. FIG. 20 shows plots of model-fitted outcome trajectories for each symptom cluster, with steeper symptom trajectories representing better outcomes. FIG. 20A shows outcome trajectories measured according to the QIDS-SR checklist in the STAR*D and CO-MED trials and FIG. 20B shows the outcome trajectories measured according to the HAM-D rating scale in 7 phase 3, placebo-controlled trials of duloxetine. The y-axes represent mean severity within a cluster and so should be multiplied by the number of symptoms within a cluster to convert to original units.

No antidepressant treatment worked equally well across all 3 symptom clusters. As shown in FIG. 20A, when measured according to the QID SSR, trajectories were significantly better for core emotional symptoms than for either sleep symptoms or atypical symptoms for citalopram, escitalopram with placebo, and escitalopram with bupropion (all |β|>0.079; all false-discovery rate corrected P<0.001). Sleep trajectories were also better than atypical trajectories for these 3 treatments (all |β|>0.099; all P≤0.001). As shown in FIG. 20B, when measured according to the HAM-D rating scale, a similar pattern was observed. Core emotional trajectories were better than sleep and atypical trajectories for all treatments (all |β|>0.12; all P≤0.001). Sleep trajectories were also better than atypical trajectories for low dose duloxetine and escitalopram (all |β|>0.080; all P≤0.001).

To interpret the magnitude of differences between drugs, an effect size (ES) was calculated, measured in raw rating scale points, that reflects the difference between treatments in reducing the overall severity of a symptom cluster (i.e., slope contrasts were multiplied by the natural log of treatment duration and then by the number of symptoms in each cluster). For example, in this study, high-dose duloxetine was significantly better than escitalopram in treating atypical symptoms, such that a patient's total improvement in atypical severity was a mean of 1.9 HAM-D points greater with high dose duloxetine than escitalopram (ES, 1.9; 95% CI, 1.4-2.3; false-discovery rate corrected P<0.001).

For each symptom cluster, there were significant differences in efficacy between treatments as shown in FIG. 20. Combined escitalopram and bupropion treatment was significantly more effective in treating core emotional symptoms than citalopram (ES, 0.7 QIDS-SR points; 95% CI, 0.2 to 1.3; P=0.03). For sleep/insomnia symptoms, venlafaxine with mirtazapine out-performed citalopram (ES, 1.4; 95% CI, 1.0 to 1.8; P<0.001). For core emotional symptoms in HAM-D scale trials (FIG. 20B), high-dose duloxetine outperformed escitalopram (ES, 2.3 HAM-D points; 95% CI, 1.6 to 3.1; P<0.001). Escitalopram was not significantly different from placebo for core emotional symptoms (ES, 0.03 HAM-D points; 95% CI, −0.7 to 0.8; P=0.94). For sleep symptoms, high-dose duloxetine outperformed fluoxetine (ES, 0.9; 95% CI, 0.1 to 1.7; P=0.046). For atypical symptoms, high-dose duloxetine outperformed all others (ES, 0.5-1.9) and escitalopram was worse than placebo (ES, 0.7; 95% CI, 0.3 to 1.1; P=0.002). Among the HAM-D studies, only two antidepressant treatments (high-dose duloxetine and paroxetine) outperformed placebo for all 3 symptom clusters.

Within STAR*D, although all models performed significantly above chance (all P<0.01), substantial variability was observed in the predictability of outcomes for each cluster. The sleep symptom cluster was the most predictable (R²=19.6%; SD, 5.0%; P<0.01) and substantially more predictable than core emotional symptoms (R²=14.5%; SD, 4.6%; P<0.01) and atypical symptoms (R²=15.1%; SD, 5.3%; P<0.01). The observed range in cluster predictability (R² difference, 5.1%) was also significantly larger than any range observed during permutation testing (mean [SD] range, 0.56% [0.50%]; P<0.01). The best predictive baseline variables were inspected for each model separately, highlighting those identified as predictive for one cluster but not others (i.e., specific predictors). Baseline HAM-D scale severity was a top predictor of core emotional outcomes but not any of the other three clusters. Baseline atypical symptom severity and hypersomnia predicted atypical outcomes; baseline sleep cluster severity and early-morning insomnia predicted sleep outcomes.

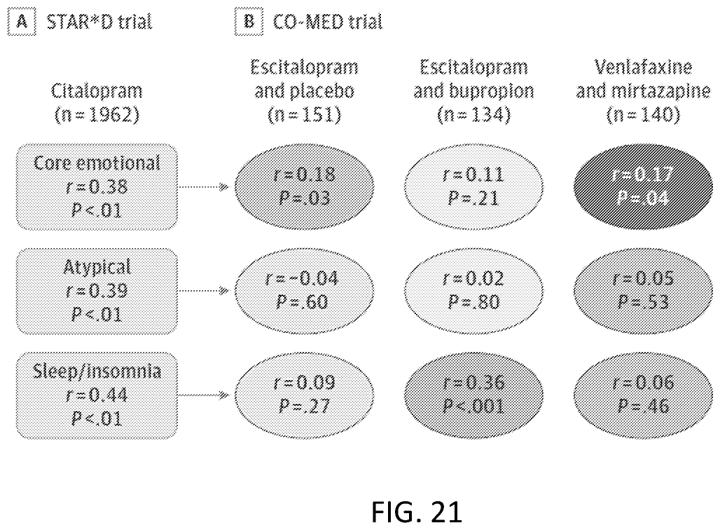

The best-performing models were then applied, without modification, to predict outcomes for each cluster in the three treatment groups of CO-MED as shown in FIG. 21. For each symptom cluster, a new model was trained on patients who received citalopram in the STAR*D cohort. Performance was statistically above chance, although clinically modest, for predicting core emotional outcomes in the escitalopram monotherapy arm (r₁₄₉=0.18; P=0.03) and the venlafaxine-mirtazapine arm (r₁₃₈=0.17; P=0.04). Performance was above chance predicting sleep outcomes in the escitalopram-bupropion arm (r₁₃₂=0.36; P<0.001).

In this example implementation of some embodiments, three symptom clusters within the QIDS-SR checklist were identified using a data-driven approach. The clustering solution was replicated in an independent trial cohort (CO-MED) and it was found to be robust across different parameters and time points and consistent with other statistical approaches. No antidepressant was equally effective for all three symptom clusters, and, for each symptom cluster, there were significant differences in treatment efficacy between drugs. Antidepressants in general worked best in treating core emotional and sleep symptoms and were less effective in treating atypical symptoms. The magnitude of these differences suggests that selecting the best drug for a given cluster may have a bigger benefit than that gained by use of an active compound vs a placebo. Treatment outcomes at the symptom cluster level were predictable by machine learning of self-report data.

The data-driven approach described herein offers some novel symptom groupings relative to previous approaches. For instance, the emotional cluster resembled the HRSD-7 subscale but never included a suicide item, and when scored according to the HAM-D scale, the HRSD-7 energy/fatigability item clustered with insomnia symptoms rather than emotional symptoms. There were slight differences between the QIDS-SR and the HAM-D scale results. In the HAM-D scale, the emotional cluster included an anxiety item, whereas in the QIDS-SR scale, the same cluster included low energy and concentration. The energy/concentration item falls in the sleep cluster for the HAM-D scale. This data-driven approach may have identified a set of symptoms in the emotional presentation of depression that may have neural circuit correlates that are more cohesive than either the DSM criteria or theory driven clusters, such as the Bech/Maier scales, which have not yet produced meaningful signatures on neural circuits or treatment response prediction.

Clusters of symptoms are detectable in two common depression rating scales, and these symptom clusters vary in their responsiveness to different antidepressant treatments. These patterns may offer clinicians evidence for tailoring antidepressant selection according to the symptoms that a specific patient is experiencing immediately—almost doubling the expected effect size of a treatment.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In this respect, it should be appreciated that one implementation of the embodiments of the present invention comprises at least one non-transitory computer-readable storage medium (e.g., a computer memory, a portable memory, a compact disk, a tape, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

1. A system for providing a treatment recommendation for a patient having a depression disorder, the system comprising:
a server computer configured to:
receive patient information including patient responses to each of a plurality of questions provided on a questionnaire;
process, with a trained statistical model, a set of values determined based, at least in part, on at least some of the received patient information to determine treatment recommendation information for the patient; and
transmit the treatment recommendation information to an electronic device.

2. The system of claim 1, wherein the set of values comprises a plurality of numerical values corresponding to features of the trained statistical model.

3. The system of claim 1 wherein the trained statistical model comprises a plurality of decision trees and a weighting function that aggregates output from the plurality of decision trees.

4. The system of claim 1, wherein the treatment recommendation information comprises a recommendation to treat the patient with one or more antidepressants selected from the group consisting of citalopram, escitalopram, and a combination of escitalopram and bupropion.

5. The system of claim 1, wherein the server is further configured to perform symptom clustering on at least some of the received patient information.

6. The system of claim 5, wherein processing the set of values with a trained statistical model to determine treatment recommendation information for the patient comprises determining the treatment recommendation information based, at least in part, on a result of performing symptom clustering.

7. The system of claim 5, wherein performing symptom clustering comprises assigning the patient to one of a plurality of symptom cluster groups.

8. The system of claim 7, wherein the symptom cluster groups comprise a sleep symptom cluster group, an emotional cluster group, and an atypical symptom cluster group.

9. The system of claim 1, wherein the server is further configured to instruct the electronic device to display a summary report of the treatment recommendation information on a user interface provided on the electronic device.

10. The system of claim 1, wherein the server is further configured to convert one or more of the received patient information to a format acceptable as input to the trained statistical model.

11. The system of claim 1, wherein an output of the trained statistical model is a plurality of values representing a percentage likelihood that a treatment will be effective for the patient, and wherein the server is further configured to determine the treatment recommendation information based on the plurality of values output from the trained statistical model.

12. The system of claim 1, wherein the server is further configured to update the trained statistical model based, at least in part, on at least some of the received patient information and at least some of the treatment recommendation information.

13. A system for training a statistical model used to predict a treatment outcome for patients having depression, the system comprising:
a server computer configured to:
determine a set of features to include in the statistical model;
train the statistical model using a first labeled dataset of values for the determined set of features, wherein the first labeled dataset includes clinical outcomes and patient information for each of a first plurality of patients; and
output the trained statistical model.

14. The system of claim 13, wherein the server is further configured to:
validate the trained statistical model using a second labeled dataset of values for the determined set of features, wherein the second labeled dataset includes clinical outcomes and patient information for each of a second plurality of patients different than the first plurality of patients.

15. The system of claim 13, wherein determining the set of features to include in the statistical model identifying the features in the set of features by applying penalized logistic regression to the data in the first labeled dataset.

16. A method of providing a treatment recommendation for a patient having a depression disorder, the method comprising:
processing using a trained statistical model executing on at least one computer processor, a set of values determined based, at least in part, on patient information including patient responses to each of a plurality of questions provided on a questionnaire;
determining treatment recommendation information based, at least in part, on output of the trained statistical model; and
transmitting the treatment recommendation information to an electronic device.

17. The method of claim 1, further comprising performing symptom clustering on at least some of the patient information.

18. The method of claim 17, wherein processing the patient information with a trained statistical model comprises processing the patient information based, at least in part, on a result of performing symptom clustering.

19. The method of claim 18, wherein performing symptom clustering comprises assigning the patient to one of a plurality of symptom cluster groups.

20. The method of claim 16, further comprising updating the trained statistical model based, at least in part, on at least some of the patient information and/or at least some of the determined treatment recommendation information.

21. A system for classifying, based on their symptoms, patients having a depression disorder into one or more symptom cluster groups, the system comprising:
a server computer configured to:
receive patient responses to each of a plurality of questions provided on a questionnaire by a plurality of patients;
apply a symptom clustering technique to the received patient responses to determine a plurality of symptom cluster groups; and
output a representation of the plurality of symptom cluster groups.

22. The system of claim 21, wherein the server computer is further configured to:
receive patient information for a patient, wherein the patient information includes patient responses to each of the plurality of questions provided on the questionnaire;
determine based, at least in part, on the received patient information, likelihood information indicating that the patient is associated with one or more of the plurality of symptom cluster groups; and
transmit the likelihood information to an electronic device.
